(19) United States
(12) Patent Application Publication
(10) Pub. No.: US 2004/0023256 A1
(43) Pub. Date: Feb. 5, 2004

(54) Translation profiling

(76) Inventors: Joseph D. Puglisi, Stanford, CA (US); Scott C. Blanchard, Palo Alto, CA (US); Ruben L. Gonzalez, Hayward, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/352,630

(22) Filed: Jan. 27, 2003

Related U.S. Application Data
(60) Provisional application No. 60/351,919, filed on Jan. 25, 2002.

(51) Int. Cl. C12Q 1/68; C07H 21/02

(52) U.S. Cl. 435/6; 536/23.1

(57) ABSTRACT

Surface-bound, translationally competent ribosome complexes are used to generate a translation profile for mRNA, which mRNA may be a single molecular species, or a combination of species, including complex mixtures such as those found in the set of mRNAs isolated from a cell or tissue. One or more components of the surface-bound ribosome complex may be labeled at specific positions to permit analysis of multiple or single molecules for determination of ribosomal conformational changes and translation kinetics. Translation profiles are used as the basis for comparison of an mRNA or set of mRNA species. The translation profile can be used to determine such characteristics as kinetics of initiation, kinetic of elongation, identity of the polypeptide product, and the like. Analysis of translation profiles may be used to determine differential gene expression, optimization of mRNA sequences for expression, screening drug candidates for an effect on translation, etc.

### STATEMENT REGARDING GOVERNMENT RIGHTS

This invention was supported at least in part by grant number GM51266 from the National Institutes of Health. The U.S. Government may have certain rights in the invention.

### BACKGROUND OF THE INVENTION

Protein synthesis is performed by the ribosome, which in conjunction with many exogenous factors translates the genetic code into protein. This process of translation has important practical aspects. The ribosome is a target for many clinically important antibiotics, and tools to monitor the ribosome and translation find use in drug screening. Translation also provides the route from gene to expressed protein.

Translation of the mRNA genetic code into protein is the final step in genetic information transfer. While current methods of gene expression analysis can determine the cellular levels of individual mRNAs, these must be assumed to correlate with the final amounts of the encoded proteins. However, in many cases translation of mRNA by the ribosome has been shown to be dependent on the sequence and structure of the mRNA. Therefore, assessment of an expression profile by looking solely at mRNA levels ignores the subtleties and regulation of translation by the ribosome. Often, translation initiation is the rate limiting step in protein synthesis; in addition, different mRNAs are translated at different rates through differences in the elongation rate of protein synthesis. Methods of screening for translation of mRNAs could provide an important means of evaluating gene expression.

The ribosome is also an important target for a wide variety of antibiotics. Many of them, such as streptomycin and tetracycline, were of great clinical importance when they were first discovered, but unfortunately strains of bacteria with resistance to these drugs have become commonplace, limiting their effectiveness. At the same time, many other antibiotics targeting the ribosome have insufficient specificity toward bacterial (as opposed to eukaryotic) ribosomes, and hence are too toxic for routine clinical use in humans. With the emergence of new multi-drug resistant strains of bacteria, there is a real need to understand details of how these antibiotics interact with the ribosome, and for screening methods to assess new drug candidates.

Many of the ribosome-directed antibiotics target rRNA, which forms critical functional sites on the ribosome. The antibiotics are thus both powerful mechanistic tools to dissect individual steps of protein synthesis, and lead compounds for the development of novel therapeutic agents. The ribosome and translation are important targets for therapeutic intervention, not only for treatment of infectious disease, but also treatment of human diseases that involve protein expression.

The rich structural information on the ribosome lies in stark contrast to knowledge of its dynamics. Systems that permit the analysis of translation are of great interest for synthetic and screening methods.

Relevant Publications

The analysis of single molecule fluorescence is disclosed in, for example, Ha et al. 1999) Proc Natl Acad Sci USA 96(3): 893-8; Ha et al. (1999) Proc Natl Acad Sci USA 96(16): 9077-82; Weiss (1999) Science 283(5408): 1676-83; and Zhuang et al. (2000) Science 288(5473): 2048-51.

The use of ribosome display is discussed, for example, by Amstutz et al. (2001) Curr Opin Biotechnol 200112(4):400-5; and by Hanes et al. (2000) Methods Enzymol 2000;328:404-30.

Ribosome structure and function are reviewed by Puglisi et al. (2000) Nat Struct Biol 7(10):855-61; and Green and Puglisi (1999) Nat Struct Biol 6(11):999-1003. Eukaryotic ribosome function is reviewed, for example, by Lafontaine et al. (2001) Nat Rev Mol Cell Biol 2(7):514-20.

### SUMMARY OF THE INVENTION

Compositions and methods are provided for analysis of protein synthesis utilizing surface-bound, translationally competent ribosome complexes. The spatial localization of this translational system permits both large scale translation procedures, and arrays of highly parallel translation reactions. These methods find use in the analysis of expressed mRNAs for their ability to produce protein; for screening individual mRNA templates for the ability to be translated into protein, for screening biological agents for their ability to enhance or interfere with translation, and the like.

In one embodiment of the invention, the surface translation system is used to generate a translation profile for mRNA, which mRNA may be a single molecular species, or a combination of species, including complex mixtures such as those found in the set of mRNAs isolated from a cell or tissue. Translation profiles can be used as the basis for comparison of an mRNA or set of mRNA species. The translation profile can be used to determine such characteristics as kinetics of initiation, kinetic of elongation, identity of the polypeptide product, and the like. Analysis of translation profiles may be used to determine differential gene expression, optimization of mRNA sequences for expression, screening drug candidates for an effect on translation, etc.

One or more components of the surface-bound ribosome complex may be labeled at specific positions to permit analysis of multiple or single molecules for determination of ribosomal conformational changes and translation kinetics. The surface bound system of the present invention allows the detection of an effect on translation from altering the translational environment, where the environment may include exogenous agents, e.g. drug candidates; mRNA sequence changes; salt concentration; pH, the presence of factors; and the like. Such methods are useful in qualitative, quantitative, and competitive assays.

### DETAILED DESCRIPTION OF THE INVENTION

Flexible multiplex screening assays are provided for the screening and biological activity classification of biologically active agents and protein coding sequences. A surface translation system is used to generate a translation profile for mRNA, which mRNA may be a single molecular species, or a combination of species, including complex mixtures such as those found in the set of mRNAs isolated from a cell or tissue. Translation profiles can be used as the basis for comparison of an mRNA or set of mRNA species. The translation profile can be used to determine such characteristics as kinetics of initiation, kinetic of elongation, identity of the polypeptide product, and the like. Analysis of translation profiles can be used to determine differential gene expression, optimization of mRNA sequences for expression, screening drug candidates for an effect on translation, etc. The measurement of translation kinetics provides highly complementary information to other methods of gene expression analysis, e.g. quantitation and differentiation of mRNA populations.

Translationally competent ribosome complexes are immobilized on a solid surface. The site of attachment is selected so as to avoid steric interference with translation, and may be accomplished through the use of a specific binding partner to ribosomal RNAs; mRNA; ribosomal proteins, and other polynucleotide or polypeptide components. A spatial array of immobilized ribosomes may be produced on a planar substrate, microbeads, on fiber optics; and the like.

One or more components of the surface-bound ribosome complex may be labeled at specific positions to permit analysis of multiple or single molecules for determination of translation kinetics. Ribosomal RNAs, including mRNA and tRNA; ribosomal proteins; and other factors and agents involved in translation may be labeled at specific positions, and arrays of immobilized ribosomes may comprise a panel of different labels and positions of labels.

Detection of the label can then be used to monitor translation kinetics, such as the initiation and elongation rats of protein synthesis. Single molecule analysis can detect rare events that are not observed in bulk, ensemble-averaged measurements, and allow heterogeneity in the system to be sorted and characterized, allowing the analysis of overall translation rates for different mRNAs bound to the surface. For multistep processes such as translation, single molecule analysis eliminates the requirement for synchronization of large numbers of molecules. Distance scales probed by methods such as fluorescence resonance energy transfer (FRET) are on the order of about 20-80 Å, which permits determination of translation kinetics. To perform single-molecule analysis of a biomolecular system, the molecules are specifically localized on a derivatized quartz surface, where the attachment to the surface allows spatial localization of the particle to the optical limit of the microscope without impairing its function.

In some embodiments of the invention, the polypeptide product is screened for function, presence of epitopes, binding, etc., by localizing the polypeptide product at or near the site of the surface bound ribosome, for example by independently binding the polypeptide to the surface, by maintaining the polypeptide bound to the ribosome, and the like.

### Translation Profile

To generate a translation profile, a test sample comprising an mRNA or set of mRNAs of interest is combined with a translationally competent ribosome complex. The ribosome complex may be bound to a surface prior to combination with the mRNA, or may be immobilized after complexing with the mRNA. At least one component of the mRNA/ribosome complex will comprise a detectable label, and preferably at least two components are separately labeled with fluorochromes that form a donor/acceptor pair for FRET. Translation kinetics, i.e. the rate of initiation of translation, and/or translation elongation, and/or translation termination can be determined through fluorescence spectroscopy of such label(s). In one embodiment of the invention, single molecule fluorescence is used to determine the translation kinetics. For example, FRET analysis of the interaction between a labeled ribosome and separately labeled mRNA can be used to determine the translation kinetics of a single mRNA molecule.

Further information may be included in a translation profile by the addition of translation kinetics from samples comprising variation in sequence, mRNA composition, and/or reaction conditions. Reactions conditions may include the addition of exogenous agents that affect translation, e.g. antibiotics; by variation in ionicity, temperature, biological factors, etc. Sequence changes can be made to the mRNA to determine, for example, the effect of codon usage, three-dimensional structure and the like on translation. Data points from two or more combinations of sequence and reaction condition can be compared, for example to a similarly obtained control sample which may be a positive or a negative control. The comparison may be a subtraction of the two values, ratio of the two, etc. Comparison can also be made against libraries of compounds, where the translation kinetics in the presence of one agent is compared to the translation kinetics in the presence of another agent, which may be unrelated, or may be related or analogous compounds.

The results can be entered into a data processor to provide a translation profile dataset. Algorithms are used for the comparison and analysis of translation profiles obtained under different conditions. The effect of sequence, factors and/or agents is read out by determining changes in translation kinetics in the translation profile. The translation profile will include the results from the test sample, and may also include one or more of the other samples as described above. A database of translation profiles can be compiled from sets of experiments, for example, a database can contain translation profiles obtained from a panel of different mRNA sequences, with multiple different changes in reaction conditions, where each change can be a series of related compounds, or compounds representing different classes of molecules.

Mathematical systems can be used to compare translation profiles, and to provide quantitative measures of similarities and differences between them. For example, the translation profiles in the database can be analyzed by pattern recognition algorithms or clustering methods, e.g. hierarchical or k-means clustering, etc., that use statistical analysis to quantify relatedness. These methods can be modified by weighting, employing classification strategies, etc. to optimize the ability of a translation profile to discriminate different functional effects.

### mRNA Test Samples

The mRNA for analysis can be prepared according to conventional methods, including isolation from cells where the cells may be prokaryote or eukaryote, e.g. freshly isolated biological samples taken from an organism, cultured cells, genetically modified cells, etc.; or the mRNA can be prepared by in vitro transcription reactions, in vitro synthesis, and the like. The mRNA can comprise a single sequence, which can be a naturally existing sequence or a genetically modified sequence. Alternatively, complex mixtures of mRNA can be evaluated, e.g. when isolated from a biological sample.

A large number of public resources are available as a source of genetic sequences, e.g. for human, other mammalian, bacterial, plant, protozoan, and animal sequences. A substantial portion of the human genome is sequenced, and can be accessed through public databases such as Genbank. Resources include the uni-gene set, as well as genomic sequences. cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo.

In some cases the mRNA will be hybridized, particularly at the 5′ end, with a labeled oligonucleotide. For example, eukaryotic mRNA can be hybridized to a labeled poly-thymidine or poly-uridine probe. Suitable hybridization conditions are well known to those of skill in the art and reviewed in Molecular Cloning: A Laboratory Manual (Sambrook et al., Cold Spring Harbor Laboratory Press, New York, 1989). Labeling of the oligonucleotide probe is performed by conventional methods known to those of skill in the art.

### Methods of Screening mRNA Test Samples

Various methods are utilized to generate a translation profile from an mRNA sample. For example, a labeled oligonucleotide may be hybridized downstream on the mRNA of choice, and the hybridized mRNA then combined with a surface bound ribosome complex, where the ribosome complex comprises a label that is a complementary donor/acceptor to the oligonucleotide label. Translation is initiated by buffer exchange with an translation extract, e.g. wheat germ, E. coli S100 extract, etc. Translation elongation is measured as appearance of a FRET signal as the labeled ribosome approaches the labeled oligonucleotide. The dye label on the ribosome can be attached to the 30S subunit, near where the 3′ end of the mRNA exits from the ribosome, e.g. the cleft near ribosomal protein S5 is the leading edge of the translating ribosome. Thus, labeling sites on the ribosome side would include a beak or H16 label, as discussed in more detail below. An alternate labeling approach utilizes reconstituted 30S particles with labeled S5 protein; a number of single-cysteine mutants of S5 have been derivatized and successfully incorporated into 30S subunits.

In one embodiment of the invention, mRNAs are isolated from cells and mRNAs undergoing translation initiation or elongation are coupled to the encoded protein undergoing synthesis via the ribosome. This is done using commercially available, small molecule antibiotic drugs, e.g. aminoglycosides, that reversibly lock down and arrest the translation apparatus thereby linking genotype and phenotype. mRNAs arrested in this manner are then isolated from the cell and hybridized to a DNA array comprising oligonucleotides complementary to downstream portions of the different mRNAs. The translation kinetics can be determined using FRET.

In another embodiment, labeled DNA oligonucleotides of from about 6 to about 20, usually about 8 to 10 nucleotides are pre-hybridized to mRNA in the test sample, where the site for hybridization is immediately downstream from the initiation codon. An initiation complex with the hybridized mRNA-DNA complex is assembled on a solid surface, and translation initiated by buffer exchange with an translation extract, e.g. wheat germ, E. coli S100 extract, etc. The labeled oligonucleotide is displaced by the ribosome when its leading edge hits the duplex, about 15 nts from the 5′-position of the A-site codon. Elongation rates are measured from the lag time until loss of fluorescence. Similarly, two labeled oligonucleotides that each comprise one member of a donor acceptor fluorochrome pair may be hybridized successively downstream of the start codon. Translation is initiated, e.g. by buffer exchange with a suitable extract, and sequential loss of fluorescence from the fluorochromes is measured.

In another embodiment of the invention, translation is initiated in the presence of a labeled oligonucleotide complementary to the region of mRNA occluded by the ribosome in the initiation complex. When sufficient polypeptide elongation has occurred to move the ribosome downstream of the initiation site, the mRNA is free to hybridize the oligonucleotide, thereby providing a signal for FRET.

An alternative method utilizes mRNA that comprises an epitope for which a high affinity antibody is available. Numerous such epitopes are known in the art, e.g. the sequence encoding the amino acid EQKLISEEDL, which is the epitope for high-affinity binding by anti-myc antibody. The epitope will be exposed to the antibody upon its emersion from the 50S subunit exit tunnel, which protects about 40-50 amino acids. Binding of labeled antibody will lead to localization of the label, which means at least about 40-50 amino acids have been synthesized. The epitope tag can be incorporated into any coding sequence of interest, and may be positioned at varying sites throughout the coding sequence. From the time lag before localization of fluorescence as a function of tag position, translation rates can be estimated. As an alternative to an epitope tag, peptide sequences that form fluorescent arsenate complexes can be inserted into the coding sequence. Translation of such modified mRNA is performed in the presence of the labeling arsenic compound.

### Candidate Agent Test Samples

Candidate agents of interest are biologically active agents that encompass numerous chemical classes, primarily organic molecules, which may include organometallic molecules, inorganic molecules, genetic sequences, etc. An important aspect of the invention is to evaluate candidate drugs for an effect on translation. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 :l to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Antibiotics

A number of clinically important drugs interfere with protein translation, and find use in the generation of translation profiles, as well as providing target molecules for modification and development of new therapeutic entities. These compounds find use in binding mRNA to ribosome complexes, for ribosome labeling purposes, for investigation of conformation and kinetics in translation, and in drug development.

Compounds of interest include aminoglycosides, which inhibit protein synthesis by irreversibly binding to 30S ribosomal subunit. Furthermore, these antibiotics interfere with human immunodeficiency virus (HIV) replication by disrupting essential RNA-protein contacts. Aminoglycosides currently in clinical use include amikacin, gentamicin, kanamycin, netilmycin, neomycin B, paromomycin, streptomycin and tobramycin. Hygromycin B is active against both prokaryotic and eukaryotic cells, and differs in structure from other aminoglycosides by having a dual ester linkage between two of its three sugar moieties resulting in a fourth, 5-membered ring. The drug works primarily by inhibiting the translocation step of elongation and, to a lesser extent, causes misreading of mRNA. In eukaryotes, the antibiotic affects EF-2-mediated translocation of A site bound tRNA to the P site, accompanied by an increase in the affinity of the A site for aminoacyl-tRNA.

Aminoglycoside antibiotics are multiply charged compounds of high flexibility. The positive charges are attracted to the negatively charged RNA backbone. The flexibility of the aminoglycosides facilitates accommodation into a binding pocket within internal loops of RNA helices or into ribozyme cores for making specific contacts. The majority of these antibiotics are composed of amino sugars linked to a 2-deoxystreptamine ring. The conserved elements among aminoglycosides are rings I and I and, within ring II, the amino groups at positions 1 and 3. These elements are essential for binding to the decoding site of the 16S rRNA. The 2-deoxystreptamine ring is substituted, most commonly, at positions 4 and 5, as in the neomycin class, or at positions 4 and 6, as in the kanamycin and gentamicin classes.

The tetracyclines inhibit protein synthesis by binding to 30S ribosomal subunit and blocking binding of aminoacyl transfer-RNA. It appears likely, however, that the initial binding of a ternary complex of EF-Tu with tRNA to the A site and the process of decoding are not affected since ribosome-dependent GTP hydrolysis by EF-Tu is unaffected by tetracycline. Tcs have no apparent effect on the binding of tRNA to the P site except during factor-dependent initiation. Consistent with the inhibition of tRNA binding to the A site during translation, Tcs also prevent binding of both release factors RF-1 and 2 during termination, regardless of the stop codon. Tetracyclines currently in clinical use include demeclocycline, doxycycline, methacycline, minocycline, oxytetracycline and tetracycline.

The macrolides inhibit protein synthesis by binding to 50S ribosomal subunits, inhibiting translocation of peptidase chain and inhibiting polypeptide synthesis. This group includes azithromycin, clarithromycin, dirithromycin and erythromycin. The lincosamide antibiotics, e.g., clindamycin and lincomycin, interfere with transpeptidation and early chain termination.

Linezolid inhibits the first step of protein synthesis by binding to f-met-t-RNA-mRNA-30s ribosome subunit. Evernimicin (Evn), an oligosaccharide antibiotic, interacts with the large ribosomal subunit and inhibits bacterial protein synthesis by interacting with a specific set of nucleotides in the loops of hairpins 89 and 91 of 23S rRNA in bacterial and archaeal ribosomes.

Pactamycin (Pct) was isolated from Streptomyces pactum as a potential new human antitumor drug, but is in fact a potent inhibitor of translation in all three kingdoms, eukarya, bacteria, and archaea. For this reason, the drug is expected to interact with highly conserved regions of 16S RNA, both structurally and with respect to sequence. In bacteria, Pct inhibits the initiation step of translation. Binding of the drug prevents release of initiation factors from the 30S initiation complex, which in turn prevents the formation of functional 70S ribosomes. The antibiotic interferes with factor and GTP-dependent binding of tRNA to the ribosomal P site during initiation, but factor-free initiation does not seem to be affected.

### Methods of Screening Candidate Agents

Samples comprising candidate agent are screened for their effect on translation, by combining the candidate agent with a surface bound translation complex comprising at least one mRNA species capable of translation by the system. Agents are screened for biological activity by adding the agent to at least one, and in some cases a plurality, of combinations of translation complexes. The change in ribosome conformation and/or translation kinetics in response to the agent is measured, desirably normalized, and the resulting translation profile may then be evaluated by comparison to reference translation profiles. The reference translation profiles may include readouts in the presence and absence of other agents, e.g. antibiotics with known action, positive controls, etc. Agents of interest for analysis include any biologically active molecule with the potential to modulate translation.

The agents are conveniently added in solution, or readily soluble form, to the medium of the surface bound ribosome complex. The agents may be added in a flow-through system, as a stream, intermittent or continuous, or alternatively, adding a bolus of the compound, singly or incrementally, to an otherwise static solution. Preferred agent formulations do not include additional components, such as preservatives, that may have a significant effect on the overall formulation.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### Surface Translation System

An array of surface bound translationally competent ribosome complexes are utilized to generate translation profiles. The array may comprise a single type of ribosome, to which can be added various exogenous agents and MRNA test samples. Alternatively the array may comprise a panel of ribosome complexes, where there is variation on the site of labels, the type of labels, the mRNA template, and the like. For example, different positions for the label allow detection of specific changes in ribosome conformation and protein synthesis. As described below, the array may be spotted on a planar surface, or present on discrete substrates, such as fibers, microspheres, etc.

The surface bound system of the present invention allows the detection of an effect on translation from altering the translational environment, where the environment may include exogenous agents, e.g. drug candidates; mRNA sequence changes; salt concentration; pH, the presence of factors; and the like. Such methods are useful in qualitative, quantitative, and competitive assays, e.g. in screening antibiotics, optimization of mRNA sequence for translation, optimization of in vitro translation conditions, etc. For example, see co-pending patent application No. 60/351,846, filed concurrently with the present application, and herewith incorporated by reference in its entirety.

Translationally competent ribosome. Ribosomes are ribonucleoprotein particles that perform protein synthesis using a messenger RNA template. The ribosome, a 70S particle in prokaryotes, is composed of two sub-units. The small subunit (30S) mediates proper pairing between transfer RNA (tRNA) adaptors and the messenger RNA, whereas the large subunit (50S) orients the 3ends of the aminoacyl (A-site) and peptidyl (P-site) tRNAs and catalyzes peptide bond formation. The ribosome translocates directionally along mRNA in 3 nucleotide steps to read the sequential codons. For the purposes of the present invention, ribosomes may be prokaryotic or eukaryotic. The term “ribosome complex” may be used herein to refer to a complex of ribosome in association with one or more biomolecules associated with translation, including, without limitation, mRNA, tRNAs, nascent polypeptide, elongation and initiation factors.

As used herein, translational competence is the ability of a ribosome to catalyze at least one peptide bond formation where the tRNA and mRNA template are properly paired, and may include the ability to catalyze translation of a complete mRNA into the appropriate protein.

It will be understood by those of skill in the art that other components may be required for translation, including, for example, amino acids, nucleotide triphosphates, tRNAs and aminoacyl synthetases, or aminoacyl-loaded tRNAs; elongation factors and initiation factors. In addition the reaction mixture may comprise enzymes involved in regenerating ATP and GTP, salts, polymeric compounds, inhibitors for protein or nucleic acid degrading enzymes, inhibitor or regulator of protein synthesis, oxidation/reduction adjuster, non-denaturing surfactant, buffer component, spermine, spermidine, etc. The salts preferably include potassium, magnesium, ammonium and manganese salt of acetic acid or sulfuric acid, and some of these may have amino acids as a counter anion. The polymeric compounds may be polyethylene glycol, dextran, diethyl aminoethyl, quaternary aminoethyl and aminoethyl. The oxidation/reduction adjuster may be dithiothreitol, ascorbic acid, glutathione and/or their oxides. Also, a non-denaturing surfactant, e.g. Triton X-100 may be used at a concentration of 0-0.5 M. Spermine and spermidine may be used for improving protein synthetic ability. Preferably, the reaction is maintained in the range of pH 5-10 and a temperature of 20°-50° C., and more preferably, in the range of pH 6-9 and a temperature of 25°-40° C.

In some embodiments of the invention, the ribosome comprises rRNA that has been genetically modified, e.g. to introduce attachment sites, sites for labeling, etc. The genetic modification can be introduced into the chromosome of the host cell from which the ribosome is derived, or more conveniently is introduced on an episomal vector, e.g. phage, plasmid, phagemid, and the like. Preferably the host cell into which the vector is introduced will lack the corresponding native rRNA genes. Ribosomes are therefore assembled using cellular machinery. The ribosomes are purified from the host cell by conventional methods known to those of skill in the art.

Substrate attachment. Translationally competent ribosomes or ribosome complexes are attached to a solid surface at a specific attachment site, where the attachment site is one of a specific binding pair. Preferably the attachment site is other than the nascent polypeptide component that is being translated. The attachment site may be naturally occurring, or may be introduced through genetic engineering. Pre-formed ribosome complexes can be attached to the surface, or complexes can be assembled in situ on the substrate. The ribosome or ribosome complex is usually stably bound to the substrate surface for at least about 1 minute, and may be stably bound for at least about 30 minutes, 1 hour, or longer, where the dissociation rate of the complexes depends on solution conditions and ligand-bound state of the ribosome. Complexes are usually more stable at higher Mg⁺⁺concentrations and monovalent ion concentrations. The complex stability may also be increased at lower pH, by the presence of a P-site tRNA, and by addition of an acyl-aminoacid on the tRNA.

In one embodiment of the invention, the attachment site is a nucleic acid sequence present in one of the ribosomal RNAs or on the mRNA, where a polynucleotide having a sequence complementary to the attachment site acts a linker between the ribosome complex and the solid surface. A convenient nucleic acid attachment site is mRNA, usually at the 5′ end, where a complementary polynucleotide may hybridize, for example, to the untranslated region of the mRNA.

Alternative nucleic acid attachment sites include rRNA regions of conserved A-form helical secondary structure where the primary sequence of the helical region is not evolutionarily conserved. Examples include surface-accessible hairpin loops, particularly those regions that are not involved in tertiary structure formation. Such regions may be identified by a comparison of rRNA sequences to determine a lack of sequence similarity. Criteria include a helix of at least about 5 nt. in length, with a non-conserved nucleotide sequence.

The surface accessible loop may serve as an attachment site, or more preferably, the rRNA will be genetically modified to expand stem loop sequences by from about 6 to about 20 nucleotides, more usually from about 8 to about 18 nucleotides. Preferred rRNA suitable for such modification is the prokaryotic 16S rRNA or the corresponding eukaryotic 18S rRNA, although the 23S and 28S rRNA may also find use.

Specific sites of interest for the introduction of a stem loop expansion for an attachment site include, without limitation, the 16S rRNA H6, H10, H26, H33a, H39 and H44 loops (Wimberly et aL (2000) Nature 407(6802):327-39). In 23S rRNA, the H9, H68 and H101 may be selected (Ban et al. (2000) Science 289(5481): 905-20).

The polynucleotide having a sequence complementary to the attachment site may be indirectly coupled to the substrate through an affinity reagent comprising two binding partners. Examples of suitable affinity reagents include biotin/avidin or streptavidin; antibody/hapten; receptor/ligand pairs, as well as chemical affinity systems. For example, the substrate surface may be derivatized with avidin or streptavidin, and a ribosome complex comprising a biotin moiety present on a complementary polynucleotide is then contacted with the substrate surface, where specific attachment then occurs.

Where the polynucleotide having a sequence complementary to the attachment site is directly coupled to the substrate, various chemistries may be employed to provide a covalent bond, including homo- or heterobifunctional linkers having a group at one end capable of forming a stable linkage to the polynucleotide, and a group at the opposite end capable of forming a stable linkage to the substrate. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP) or 4-(N-maleimidomethyl)-cyclohexane-1-carboxylic acid N-hydroxysuccinimide ester (SMCC). To improve the stability, the substrate may be functionalized to facilitate attachment. Modes of surface functionalization include silanization of glass-like surfaces by 3-aminopropyltriethoxysilane, 3-mercaptopropyltrimethoxysilane, 3-isocyanatopropyltriethoxysilane, 3-isothiocyanonatopropyltriethoxysilane, 2-(4-chlorosulfonylphenyl) ethyltrimethoxysilane, 3-bromopropyltrimethoxysilane, methacryloxymethyltrimethylsilane; and the like. Polymer coating may be achieved with polyvinyl alcohol, polyethyleneimine, polyacrolein, polyacrylic acid, etc.

An alternative attachment strategy utilizes ribosomal proteins, which may be modified to include a site for biotinylation, or other binding moieties.

By “solid substrate” or “solid support” is meant any surface to which the ribosome or ribosome complexes of the subject invention are attached. Where the ribosome is labeled, preferred substrates are quartz. Other solid supports include glass, fused silica, acrylamide; plastics, e.g. polytetrafluoroethylene, polypropylene, polystyrene, polycarbonate, and blends thereof, and the like; metals, e.g. gold, platinum, silver, and the like; etc. The substrates can take a variety of configurations, including planar surfaces, beads, particles, dipsticks, sheets, rods, etc.

In one embodiment of the invention, the substrate comprises a planar surface, and ribosomes or ribosome complexes are attached to the surface, e.g. in a solid or uniform pattern, or in an array in a plurality of spots. The density of attached particles on the substrate will be such that a signal from a label can be detected. Where the complexes are spotted on the array, the spots can be arranged in any convenient pattern across or over the surface of the support, such as in rows and columns so as to form a grid, in a circular pattern, and the like, where generally the pattern of spots will be present in the form of a grid across the surface of the solid support. The total number of spots on the substrate will vary depending on the sample to be analyzed, as well as the number of control spots, calibrating spots and the like, as may be desired.

In another embodiment, the substrate is a collection of physically discrete solid substrates, e.g. a collection of beads, individual strands of fiber optic cable, and the like. Each discrete substrate can have complexes distributed across the surface or attached in one or more probe spots on the substrate. The collection of physically separable discrete substrates may be arranged in a predetermined pattern or may be separated in a series of physically discrete containers (e.g., wells of a multi-well plate).

Labeling strategies. In a preferred embodiment of the invention, one or more components of the ribosome complex comprise a fluorescent label. Suitable components include tRNAs, ribosomal proteins, elongation factors, mRNA, ribosomal RNAs, and analogs thereof, such as antibiotics that specifically bind the complex. The label may provide single molecule fluorescence, where the signal from a single fluorochrome is detected; or energy transfer, e.g. fluorescence resonance energy transfer (FRET), where a pair of fluorescent molecules interact to provide a signal. Similar experiments can be performed on large numbers of ribosomes in bulk solution.

Fluorescent labels of interest include: fluorescein, rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N, N, N′, N′-tetramethyl-6-carboxyrhodamine (TAMRA), the cyanine dyes, such as Cy3, Cy5, Alexa 542, Bodipy 630/650, fluorescent particles, fluorescent semiconductor nanocrystals, and the like.

FRET occurs when a suitable fluorescent energy donor and an energy acceptor molecule are in close proximity to one another. The excitation energy absorbed by the donor is transferred non-radiatively to the acceptor which can then further dissipate this energy either by fluorescent emission if a fluorophore, or by non-fluorescent means if a quencher. A donor-acceptor pair comprises two fluorophores having overlapping spectra, where the donor emission overlaps the acceptor absorption, so that there is energy transfer from the excited fluorophore to the other member of the pair. It is not essential that the excited fluorophore actually fluoresce, it being sufficient that the excited fluorophore be able to efficiently absorb the excitation energy and efficiently transfer it to the emitting fluorophore.

The donor fluorophore is excited efficiently by a single light source of narrow bandwidth, particularly a laser source. The emitting or accepting fluorophors will be selected to be able to receive the energy from the donor fluorophore and emit light. Usually the donor fluorophores will absorb in the range of about 350-800 nm, more usually in the range of about 350-600 nm or 500-750 nm, while the acceptor fluorophores will emit light in the range of about 450-1000 nm, usually in the range of about 450-800 nm. The transfer of the optical excitation from the donor to the acceptor depends on the distance between the two fluorophores. Thus, the distance must be chosen to provide efficient energy transfer from the donor to the acceptor.

The fluorophores for FRET pairs may be selected so as to be from a similar chemical family or a different one, such as cyanine dyes, xanthenes or the like. Reporter, or donor, dyes of interest include: fluorescein dyes (e.g., 5-carboxyfluorescein (5-FAM), 6-carboxyfluorescein (6-FAM), 2′,4′, 1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE)), cyanine dyes such as Cy5, dansyl derivatives, and the like. Acceptor dyes of interest include: rhodamine dyes (e.g., tetramethyl-6-carboxyrhodamine (TAMRA), and tetrapropano-6-carboxyrhodamine (ROX)), DABSYL, DABCYL, cyanine, such as Cy3, anthraquinone, nitrothiazole, and nitroimidazole compounds, and the like.

Specific sites of interest for labeling include tRNA, which may be labeled on the RNA or the amino acid portion of the molecule. Body labeling of the RNA itself can be accomplished, for example by synthesizing the tRNA with an amino linker, which can be derivatized. Suitable sites include the anticodon stem loop, the elbow region and 3′acceptor arm. Alternatively, the amino acids used to charge the tRNA can be labeled and then used to charge the tRNA with the appropriate aminoacyl synthetase.

Many proteins involved in the process of translation can be labeled, including ribosomal proteins, elongation and initiation factors, and the like. For example, the S21 protein sits in the tRNA exit site of the ribosome (E site), and can be dye labeled by any conventional method. The labeled protein is separated from the unbound dye, and then incubated with the suitable ribosomal subunit at a molar excess of protein to favor exchange of the native protein with the labeled protein.

Direct fluorescent labeling of ribosomal RNA can utilize a complementary polynucleotide probe that is complementary to a target sequence, where a labeled polynucleotide specifically hybridizes to a rRNA sequence. Target sites on the rRNA for hybridization include regions of conserved A-form helical secondary structure where the primary sequence of the helical region is not evolutionarily conserved. Examples include surface-accessible hairpin loops, particularly those regions that are not involved in tertiary structure formation. Such regions may be identified by a comparison of rRNA sequences to determine a lack of sequence similarity. Criteria include a helix of at least about 5 nt. in length, with a non-conserved nucleotide sequence.

The native sequence may serve as a target site, or more preferably, the rRNA will be genetically modified to expand stem loop sequences by from about 6 to about 20 nucleotides, more usually from about 8 to about 18 nucleotides. Preferred rRNA suitable for such modification is the prokaryotic 16S rRNA or the corresponding eukaryotic 18S rRNA, although the 23S and 28S rRNA may also find use. Specific sites of interest for the introduction of a stem loop expansion for an attachment site include, without limitation, the 16S rRNA H6, H10, H16, H17, H26, H33a, H33b, H39 and H44 loops. In 23S rRNA, the H9, H38, H68 H69, H72, H84, H89, H91 and H101 may be selected.

Alternatively ribosomes may be labeled using a peptide tagging strategy. The BIV Tat protein binds to a specific sequence in the context of an A-form helix with a single-nucleotide bulge; the peptide binds with high affinity (Kd nM) and specificity within the major groove of the helix. See, for example, Campisi et al. (2001) EMBO J 20(1-2):178-86. Target sites, as described above for hybridization labels, can be genetically modified to contain a BIV Tat binding site, to which is bound fluorescently labeled BIV Tat. The recognition sequence for BIV Tat is 5′ NUGNGC 3′; 5′ GCNCN 3′, where the two strands pair to form a quasi A form paired helix with a single bulged uridine; and where the N-N pair must be a Watson-Crick pair for stability. The BIV Tat peptide generally comprises the amino acid sequence RGTRGKGRRI for high binding affinity. An alternate peptide tag is the HIV Rev peptide, which binds to a purine-rich internal loop in an RNA helix. For double labeling of different subunits, the individual subunits can separated and labeled independently, using combinations of one or more peptide and/or hybridization tags.

Labeled peptide or polynucleotide probes can be synthesized and derivatized with a fluorescent tag. The labeled probes can then be incorporated into cell growth media, or bound to the ribosomes post-synthetically. When bound to the ribosome during synthesis the probes further provide a means investigating the in vivo process of ribosome assembly.

Another approach for rRNA labeling utilizes internal incorporation of dyes by ligation of 16S rRNA fragments that contain dyes at their 5′ or 3′ termini. For example, 16S rRNA can be transcribed as two pieces, with a dye-labeled dinucleotide as primer of transcription. The two strands are then ligated by DNA ligase and a DNA splint. The 30S subunit is then reconstituted from total 30S proteins using standard protocols.

### Detection and Data Analysis

Methods of fluorescence detection are known in the art. The detection element may include photodiodes, phototransistors, and photomultipliers, but is not limited thereto. The signal is then transmitted to a suitable data processor. For single molecule experiments, the internal reflectance (TIR) microscope allows simultaneous detection of hundreds of single molecules, with a time resolution of 100 ms. The fluorescent samples are excited by the evanescent wave generated by total internal reflection of dual laser excitation. Fluorescence is detected using a CCD camera, after the radiation has passed through a dichroic filter.

In the scanning confocal microscope, fluorescence is dual excited and detected using avalance photodiodes. In this instrument, the fluorescence of a single molecule, as opposed to a field of molecules, as in the TIR microscope, is monitored with a time resolution of 1 ms.

The readout may be a mean, average, median or the variance or other statistically or mathematically derived value associated with the measurement. The parameter readout information may be further refined by direct comparison with a corresponding reference readout. The absolute values obtained for each parameter under identical conditions will display a variability that is inherent in biological systems.

The comparison of a translation profile obtained from a test compound, and a reference translation profile(s) is accomplished by the use of suitable deduction protocols, Al systems, statistical comparisons, etc. The translation profile may be compiled and compared with a database of reference translation profiles. These databases may include reference translation profiles from known mRNA sequences, from defined biological samples, from assays performed in the presence of defined biological agents, and the like.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

This invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “and”, and “the” include plural referents unless the context clearly dictates otherwise. Thus, for example, reference to “a specific binding pair” includes a plurality of such specific binding pairs and reference to “the complementing domain” includes reference to one or more complementing domains and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed. All publications mentioned herein are incorporated herein by reference to disclose and describe the methods and/or materials in connection with which the publications are cited.

### Experimental

To perform single-molecule spectroscopic analysis of a biomolecular system, the molecules are specifically localized on a derivatized quartz surface. The attachment to the surface allows spatial localization of the particle to the optical limit of the microscope without impairing its function.

### EXAMPLE 1

### Ribosome Characterization

To characterize ribosomes using biophysical analysis, their chemical composition must be determined; ribosomes can be missing certain proteins (especially L7/L12) that decrease activity, or rRNA can be degraded. 70S ribosomal particles were purified from E. coli; subunits were dissociated and purified by sucrose density gradient centrifugation. The composition of the ribosomes was analyzed by gel electrophoresis. The RNA components (23S, 16S and 5S RNA) were all intact and present stoichiometrically. The protein composition was determined by two-dimensional electrophoresis; all 54 proteins were present. The presence of proteins most often present in sub-stoichiometric quantities, L7/L12 and S1, were monitored by native gel analysis of the ribosomal particles. The 30S subunit has different mobility plus or minus S1; likewise the 50S subunit has different mobility plus or minus L7/L12. It was shown that protein L7/L12 is present in stoichiometric amounts as a tetramer, whereas S1 is present in sub-stoichiometric ratios. Both proteins can be overproduced in the appropriate bacterial strains. The activity of the ribosome preps were checked using in vitro translation of gene 32 protein under standard conditions; the ribosomes showed appropriate activity in translation. These results demonstrate that ribosomes of defined composition can be prepared for further analysis.

### EXAMPLE 2

### Specific Surface Attachment of Ribosomes

Ribosomes can be specifically attached to quartz surfaces. Microscope slides were derivatized to provide a surface with streptavidin molecules on the surface. To detect ribosomal particles, 50S subunits were non-specifically labeled with Cy3 NHS esters, which react with surface-accessible amino groups. An average of 1 dye molecule per subunit was estimated using single-molecule fluorescence. A quaternary complex was then formed with 70S particles that have labeled 50S subunits, tRNAfMet, a short mRNA that corresponds to the first 3 codons of the gene 32 protein mRNA and a 18 nt DNA complementary to the 5′ end of the mRNA. Two complexes were formed with the DNA either 3′ biotinylated or not. The quarternary complexes were purified using a sucrose gradient and isolated. Ribosomal complexes at a concentration of 1 μM were flowed onto the quartz surface and then washed in buffer. Only ribosomal complexes with biotinylated DNA attach to the quartz surface. Cy3 fluorescence was monitored; localized spots showed that 50S subunits are localized. Since the ribosomal complexes are held to the surface by interaction between the P-site tRNA and mRNA, the presence of labeled 50S subunits means the entire complex has bound to the surface. The complexes are reversibly bound to the surface, as treatment with 50 mM EDTA releases the 50S subunits.

The 70S complexes were stably bound to the surface for minutes to hours. The dissociation rate of the complexes depends on solution conditions and ligand-bound state of the ribosome. A matrix of conditions was investigated to determine the stabilities of surface-bound ribosomal complexes. It was found that complexes are more stable at higher Mg2+concentrations and monovalent ion concentrations. This is consistent with the stabilization of RNA-RNA interactions at the subunit interface. The complex stability also increased at lower pH. Complex stability was also greatly increased by the presence of a P-site tRNA, and further increased by addition of an acyl-aminoacid on the tRNA.

Binding of transfer RNA within the surface-bound complexes was analyzed by co-localization of fluorescently-labeled tRNA with fluorescently-labeled ribosomes. Initiator tRNAfMet was methionylated by MetRS, and the free amino group of the Met-tRNAfMet was derivatized with Cy5 using NHS ester chemistry. Cy5-methionyl-tRNAfMet was purified by HPLC and complexes with Cy3-labeled 70S subunits (50S subunit labeled) mRNA and DNA were formed and purified by sucrose gradient centrifugation. These complexes were bound to the surface and Cy3 and Cy5 fluorescence was monitored. It was estimated that a lower limit of 35% of Cy3-labeled ribosomes have Cy5 tRNA bound; the low P-site occupancy may be increased by addition of increased tRNA concentration, but more likely results from hydrolysis of the aminoacyl-tRNA during complex formation. The advantage of single-molecule analysis can be seen here, as bulk measurements can not catalog ribosomes in this manner.

The surface-attached ribosomes are active in catalyzing peptide bond formation. The Cy5 tRNA complexes discussed above were used to test peptidyl transfer activity using the puromycin reaction. Puromycin is analog of aminoacyl tRNA, and binds to the A-site on the 50S subunit; it reacts to form a peptidyl-puromycin adduct that can no longer undergo chain elongation. With the complexes described above, puromycin reacts to form Cy5-met-puromycin, which is weakly bound by the ribosome and rapidly dissociates. Loss of Cy5 spots was examined as a function of time after addition of puromycin; Cy3 fluorescence was monitored simultaneously to assure that ribosomes do not dissociate during the time course of the experiment. Puromycin clearly causes release of Cy5 dye, and ribosomes are stable during the course of the experiment. The data are corrected for the rates of photobleaching of Cy5, which is insignificant on the time scale of the experiment, using shuttered excitation. All Cy5-tRNA reacts in this assay, and the rates of reaction correspond to previously measured rates for the puromycin reaction measured in bulk using biochemical methods.

The puromycin reaction on the surface is sensitive to solution conditions in a manner consistent with data from bulk measurements in solution. The rate of the peptidyl transferase reaction increases with increasing pH, as observed in bulk. This is consistent with a base-catalyzed reaction. The surface-based peptidyl transfer reaction is inhibited by antibiotics that inhibit peptidyl transfer. Chloramphenicol is a peptidyl transferase inhibitor that is a competitive inhibitor of the puromycin reaction. Addition of 1 mM chloramphenicol leads to the appropriate inhibition of the surface-based puromycin reaction. Acetyl-puromycin, which has its reactive amino group blocked by acetylation, does not lead to Cy5 release.

### EXAMPLE 3

### Labeling of Ribosomal Components and Liqands with Fluorescent Dyes

Labels were incorporated into (a) tRNAs, (b) ribosomal proteins, and (c) ribosomal RNA. For tRNA ligands, fluorescent dyes were incorporated on the amino acid of methionyl-tRNAfMet. tRNAs were also synthesized with a single amino linker that can be derivatized by NHS-ester chemistry. This has allowed body labeling tRNAs at critical functional sites, like the anticodon stem loop, the elbow region and 3′ acceptor arm.

Ribosomal protein S21, which contains a single cysteine, was labeled. The S21 was labeled initially with maleamide tetramethylrhodamine; dye labeled protein was separated from unlabeled protein by HPLC. The labeled S21 was incubated with 30S subunits at high salt and 10-fold excess S21 to favor exchange of bound S21 for labeled S21. Complexes with tRNA and mRNA were assembled as described above using unlabeled 50S subunits. This lead to surface-bound complexes with single dye molecules attached to the ribosome. The intensity of observed rhodamine fluorescence is uniform for individual spots. Thus, ribosomal proteins can be labeled and incorporated into 70S particles.

A fluorescent label was incorporated in the heart of the A site of the 50S subunit. 5′ 4sTCC-puromycin is an A-site substrate, which binds with higher affinity puromycin, due to additional ribosome contacts with C74 of tRNA. Upon radiation with light of 320 nm, 4sTCC-puromycin forms a cross link with G2553 in the A loop of 23S rRNA. This cross-linked puromycin is competent to perform the peptidyl transferase reaction. Cross-linking an oligonucleotide version of the cross-linking reagent, allows formation of a duplex with a 3′-Cy3 or Cy5 labeled oligonucleotide. Cross linking was performed, and complexes with unlabeled tRNA and non-specifically labeled 70S subunits were formed and purified by sucrose gradient centrifugation. Biochemical analysis localized the cross link to G2553, as predicted from prior studies. Single-molecule fluorescence analysis showed co-localization of the cross-linked fluorescent duplex with ribosomes; intensities were consistent with a single fluorophore per ribosome, and a cross linking efficiency of about 10%. These data show that rRNA dye labeling in active sites is possible.

Labeled S21, which binds in the E site, was used as a FRET partner for translocation of P-site tRNA towards the E-site. S21 was labeled at C21 as described above and tRNAfMet was labeled at the elbow in the D loop. S21 protein has been overexpressed and purified. An 15N-labeled sample was prepared; and a 1H-15N HSQC of the amide region determined. The dispersion of the spectrum was consistent with a weakly alpha helical structure, as supported by structure prediction and CD spectra.

RNA oligonucleotides were synthesized using in vitro transcription with T7 RNA polymerase. To avoid RNA heterogeneity, ribozyme cleavage sites were engineered at the 3′ and 5′ end of the RNA. T7 polymerase for large-scale transcription was obtained in-house by an overexpression system. RNA was purified using preparative gel electrophoresis. RNA oligonucleotides with modified nucleotides, in particular 5-alkyl amino pyrimidines, were purchased from commercial sources and purified in-house.

### EXAMPLE 4

### Ribosome Preparation, Purification, and Labeling

E. coli MRE600 cells are grown to early log phase, and then rapidly cooled to 0° C. by pouring over ice, to preserve polysomes. Cells are pelleted and lysed by lysozyme/freeze thaw-fracture method. Cell debris is removed by initial slow spin, and then ribosomes are pelleted from the supernatant by 100Kxg spin. To improve selection of active ribosomes, polysomes are separated from ribosomes and subunits by gel filtration; the isolated polysomal ribosomes are dialyzed against low Mg2+buffer to dissociate polysomes and 70S particles to subunits. Isolated subunits are purified by sucrose density gradient centrifugation; Subunits can be stored at −80° C.

Gel electrophoretic analysis of ribosomal proteins and particles. Native gels are run using a modification of published procedures (Dahlberg et al. (1969) J Mol Biol 41(1): 139-47). 2.75% polyacrylamide/0.5% agarose is the standard gel matrix. The gel buffer and running buffers are 25 mM Tris-acetate, 6 mM KCI, 2 mM MgCl, 1 mM DTT. 1% w/v sucrose is added to the gel matrix. Gels are run in the cold room with buffer recirculation and continuous cooling at 1° C. Two dimensional gel electrophoresis of ribosomal proteins is performed with a the Bio-Rad protean II xi 2d electrophoresis system using published protocols (Agafonov et al. (1999) PNAS 96(22): 12345-9).

Mutant Ribosomes. Mutations are incorporated into either low or high copy plasmids for expression of ribosomes with mutant subunits, using standard protocols, Recht et al. (1999) J. Mol. Biol. 262: 421-436. Mutations with non-lethal phenotypes can be expressed from high copy plasmids, and can be expressed as a pure population using an E. coli strain in which all 7 copies of the rRNA operon has been deleted (Asai et al. (1999) PNAS 96(5): 1971-6). Mutations that confer lethal phenotypes must be expressed using a repressed plasmid system; expression of the mutant ribosomal RNA upon induction can lead to mutant ribosomes as 20-40% of the total population of ribosomes.

Protein expression and purification. Protein expression strains are available for the following proteins: EF-Tu, EF-G, cysteine (−) mutant, his-tagged; EF-G, cysteine (−) mutant, C301 mutation, his-tagged; EF-G, cysteine (−) mutant, C506 mutation, his-tagged; EF-G, cysteine (−) mutant, C585 mutation, his-tagged; S1, S21, IF1, IF3, RRF, L7/L10 (co-expressed), L7/L10 (co-expressed): L7 C37, L7/L10 (co-expressed): L7 C63, L7/L10 (co-expressed); L7 C58, L10, L10 deletion mutant that binds only one dimer of L7; Methionyl tRNA synthase, Transformylase. Proteins are overexpressed in E. coli. Purification follows standard methods. For His-tagged proteins, a single Ni column is sufficient. For untagged proteins, multicolumn purification using FPLC is performed.

tRNA aminoacylation. Deacylated tRNAs fMet, Phe and Lys can be purchased, for example from Sigma. tRNAfMet is aminoacylated using purified MetRS; Aminoacylation is performed on large scale using 20 μM tRNA in standard aminoacylation buffers. Aminoacylated tRNA is purified from non-acylated tRNA using HPLC. Other tRNAs are aminoacylated using a mixture of E. coli aminoacyl-tRNA synthetases

Dye Coupling. Cy3 (Max 550 nm, emission max 570 nm) and Cy5 (Max 649 nm, emission max 670 nm) are purchased as either N-hydroxy-succinimyl (NHS) esters or maleimides with 6 carbon linkers (Amersham-Pharmacia); amino groups are derivatized using NHS ester chemistry, whereas —SH groups are derivatized with maleimide chemistry. For dye labeling of the NH2 group of methionyl-tRNAfMet, the reaction is performed in 100 mM triethanolamine hydrochloride in 80% v/v DMSO the final pH of the solution is 7.8. tRNA is soluble in this solution up to 100 pM and dyes are added to this solution up to 4 mM final concentration. The reaction takes place at 37° C. for 8-10 hrs and is quenched by ethanol precipitation. Free dyes are removed from tRNA by spin-column gel filtration and the desired product is easily isolated in pure because the dye molecule retards migration by more than 40 minutes from the unlabeled tRNA by HPLC. As an example of cysteine labeling, S21 is efficiently labeled with maleimide containing compounds in 7 M Guanidinium Chloride/10 mM K-Hepes pH 6.5/2 mM TCEP (a non-sulfur based reducing agent) by incubation 4° C. overnight in the presence of a 50x molar excess of labeling reagent (Cy3/5-maleimide from Amersham-Pharmacia). Before modification, the cysteine is reduced in 20 mM DTT 37° C. and gel filtered into incubation buffer Free dye is separated from S21 using cation exchange resin. Coupling efficiency is monitored by gel electrophoresis.

Ligation of RNAs. RNAs are ligated on large scale using T4 RNA ligase; we have achieved ligation efficiencies on large scale of 10-50%. To avoid self ligation, the 3′ strand contains both 5′ and 3′ phosphate. The 3′ phosphate is generated by transcription and hammerhead ribozyme cleavage at the 3′ end. The 5′ strand has a 3′ OH (a 5′ OH is preferable also to avoid self ligation. Ligation reactions are performed in standard ligase buffer at RNA concentrations of 50-100 μM; RNA strand concentrations, Mg2+concentration and polyethylene glycol concentrations are optimized on small-scale reaction for each sequence. We have used these ligation methods on large RNAs rich in secondary structure, such as tRNA and the HCV IRES. Three-way ligations will be performed in a stepwise manner.

Single-molecule Fluorescence Spectroscopy. Single molecule fluorescence spectroscopy is a powerful means of monitoring conformational dynamics of complex biological systems. Single molecule analysis can detect rare conformational events that are not observed in bulk, ensemble-averaged measurements. It allows heterogeneity in the system to be sorted and characterized; this is particularly important in complex, multifactor processes such as translation. For multistep processes such as translation, single molecule analysis eliminates the requirement for synchronization of large numbers of molecules. The time resolution of the single molecule fluorescence instrumentation (from 1-100 ms) is ideal to deal with the relatively slow processes of translation. The distance scales probed by fluorescence resonance energy transfer (FRET) (20-80 Å) are appropriate for the large size (250 Å) of the ribosomal particle.

The internal reflectance (TIR) microscope allows simultaneous detection of hundreds of single molecules, with a time resolution of 100 ms. The fluorescent samples are excited by the evanescent wave generated by total internal reflection of dual laser excitation (532 nm and 635 nm). Fluorescence is detected using a CCD camera, after the radiation has passed through a dichroic (635 nm longpass) filter; cy3 and cy5 emission is measured on two halves of the CCD. In the scanning confocal microscope, fluorescence is dual excited at 532 and 635 nm and detected using avalance photodiodes. In this instrument, the fluorescence of a single molecule (as opposed to a field of molecules, as in the TIR microscope), is monitored with a time resolution of 1 ms. This instrument is used for rapid kinetic measurements, as most critical conformational steps in translation occur more slowly than 1 ms. For both instruments, laser powers are 0.3-0.5W. The instruments are controlled, and data are processed using in-house software.

What is claimed is:

1. A translationally competent ribosome complex bound to a solid surface at a specific attachment site on said ribosome complex, and further comprising a fluorescent label.

2. The ribosome complex according to claim 1, further comprising a polypeptide encoded by an mRNA present in said complex.

3. The ribosome complex of claim 2, wherein said polypeptide is bound to said complex.

4. The ribosome complex of claim 1, further comprising a candidate agent bound to said complex.

5. The ribosome complex of claim 4, wherein said candidate agent is a ribosome acting antibiotic.

6. A method for preparing a translation profile for an mRNA species, the method comprising:
combining an mRNA species with a translationally competent ribosome complex according to claim 1;
initiating translation;
detecting changes in fluorescence during translation; and
correlating said changes in fluorescence with reaction kinetics.

7. The method according to claim 6, wherein said mRNA comprises a fluorescent label acting as a donor/acceptor pair with the fluorescent label present on said ribosome complex.

8. The method according to claim 7, wherein said detecting step comprises detecting a change resulting from fluorescence resonance energy transfer.

9. The method according to claim 8, wherein said detection comprises detecting fluorescence from a single molecule.

10. The method according to claim 8, wherein said detecting comprises detecting fluorescence in a bulk assay.

11. The method according to claim 6, wherein said translation profile comprises data obtained from the detection of translation initiation.

12. The method according to claim 6, wherein said translation profile comprises data obtained from the detection of translation elongation.

13. The method according to claim 6, wherein said translation comprises data obtained from the detection of translation termination.

14. The method according to claim 6, wherein a plurality of said ribosome complexes are present on an array.

15. The method according to claim 14, wherein said mRNA is a complex mixture of mRNA species.

16. The method according to claim 6, further comprising contacting a candidate biologically active agent with said ribosome complex;
comparing the measurement of translation kinetics with a measurement from a control sample.

17. The method according to claim 16, wherein said candidate biologically active agent is a ribosomal acting antibiotic.

18. The method according to claim 6, further comprising contacting a known ribosomal acting compound with said ribosome complex;
comparing the measurement of translation kinetics with a measurement from a control sample lacking said ribosomal acting compound.
